(19) United States
(12) Patent Application Publication
(10) Pub. No.: US 2009/0117207 A1
(43) Pub. Date: May 7, 2009

(54) Methods and compositions for treatment of poison-caused pathology

(76) Inventors: Csaba K. Zoltani, Lutherville, MD (US); Gennady E. Platoff, Millersville, MD (US); Steven I. Baskin, Bel Air, MD (US)

Correspondence Address: U S ARMY RESEARCH LABORATORY;ATTN AMSRL CS CC IP, 2800 POWDER MILL RD, ADELPHI, MD, 207831197, US

(21) Appl. No.: 11/288,268

(22) Filed: Nov. 29, 2005

(51) Int. Cl. A61K 31/185; A61K 31/16; A61K 31/138; A61K 31/44; A61K 31/195; A61K 33/00; A61K 33/04; A61P 9/00; A61K 31/4375

(52) U.S. Cl. 424/699; 514/710; 514/625; 514/648; 514/352; 514/567; 424/708; 424/718; 514/279

(57) ABSTRACT

A method of treating a poison-caused pathology in an individual subject is described which includes administering a pharmaceutical composition including a therapeutically effective amount of a chloride current modulator, a therapeutically effective amount of an exogenous oxygen carrier, or a combination of a therapeutically effective amount of a chloride current modulator and a therapeutically effective amount of an exogenous oxygen carrier, to an individual subject exposed to a poison. Optionally, an inventive method further includes administering a therapeutic agent to inhibit poison-caused pathology. A composition according to the invention is described which includes a therapeutically effective amount of a chloride current modulator, a therapeutically effective amount of an exogenous oxygen carrier, or a combination of a therapeutically effective amount of a chloride current modulator and a therapeutically effective amount of an exogenous oxygen carrier. Further included in one embodiment is a therapeutic agent to inhibit poison-caused pathology.

### GOVERNMENT INTEREST

The invention described herein may be manufactured, used, and licensed by or for the United States Government.

### FIELD OF THE INVENTION

This invention relates to compositions and methods for treatment of poisoning. In particular, the invention relates to compositions and methods for treatment of cardiac arrhythmias and/or repolarization abnormalities resulting from poisoning.

### BACKGROUND OF THE INVENTION

Historically, poisoning has been of medical interest in the context of deliberate poisoning during warfare, execution, and intentional ingestion or absorption. Accidental ingestion or absorption of a poison is also a significant medical issue.

In addition, poisons have significant uses in industry. Toxic agents such as cyanide are used in production of nylon, fumigants and pesticides, as well as in metal-related industries, such as ore extraction and metal plating, such that occupational exposure is a health concern. Further, cyanides are also found in various foods, such as in the kernels of several fruits, beer, tobacco and cassava, and accidental poisonings have been known to occur due to overindulgence in particular foods or improper processing of those foods.

Some of the most serious consequences of toxic agent poisoning are due to effects on the cardiovascular system. For instance, cyanide-caused heart failure is a syndrome characterized by left ventricular dysfunction, reduced exercise tolerance, impaired quality of life and dramatically shortened life expectancy. Decreased contractility of the left ventricle leads to reduced cardiac output with the consequent systemic arterial and venous constriction.

Cardiovascular effects of cyanide poisoning are apparent when analyzed by electrocardiogram. The electrocardiogram of an affected individual shows changes in the P-wave, the depolarization of the atria, and also prominent modulation of the T-wave, the repolarization of the ventricles. The resting potential rises and Brugada-like symptoms are in evidence. After an initial bradycardia, Torsade de Pointes and tachycardia develop and the electrophysiology of the heart loses its predictability. Cyanide-caused ventricular fibrillation culminates in heart failure and is recognized as the most common cause of mortality in cases of cyanide poisoning.

Effects of cyanide compounds on cardiac tissue are demonstrated in FIG. 1 which shows a simulated electrocardiogram (ECG or EKG) illustrating a control trace, A, representative of normal cardiac activity, and a trace B illustrating differences due to cyanide. A normal T-wave is shown in the simulated control trace, A, at (a). Notable changes in trace B, showing simulation of cyanide effects, include a decrease in amplitude of the QRS and the modulation of the T-wave, the repolarization of the ventricle. In particular, a T-wave as a separate entity is no longer in evidence, the shape of the QRS portion has been altered and the peak has been shortened.

Treatment of cyanide poisoning has focused predominantly on clearance of cyanide compounds from the body. Particular treatments include administration of sodium thiosulfate, thiosulfate, nitrates, such as sodium nitrate, 4-dimethylaminophenol (4-DMAP) and hydroxocobalamin (vitamin B-12). In addition, dicobalt edetate has been shown to have some efficacy in combating cyanide toxicity. A difficulty with existing methods of treating cyanide poisoning is their common reliance on clearing of the cyanide from the system of an affected individual. Such treatment takes time and, in general, especially with high dosages of cyanide, an affected individual has usually less than 10 minutes in order to reduce the cyanide levels in the system. In cyanide toxicity cases, therapy is preferably started immediately, sometimes referred to as the “three minute solution.”

Nanocapsulated cyanide metabolizing enzymes with catalytic activity have been used in therapy. One version contains murine erythrocytes with rhodanese and sodium thiosulfate that shows some promise in lowering blood cyanide concentrations. However, metabolites of cyanide, such as ATCA, are thought to play an important role in expressing cyanide toxicity, especially in the heart. Therefore, due to the low rhodanese activity, thiocyanate formation is blocked. [Brian A. Logue, Bryon J. Pieper, Ilona Petrikovics, Matthew A. Moser, and Steven I. Baskin. Paper ANYL 100, The 227th ACS National Meeting, Anaheim, Calif., Mar. 28-Apr. 1, 2004.]

Thus, there is a continuing need for new treatments for poisoning and for treating conditions relating to poison exposure. In particular, treatments addressing cardiac sequelae of cyanide exposure and other types of poisoning having similar cardiac effects are especially sought after.

### SUMMARY OF THE INVENTION

A method of treating a poison-caused pathology in an individual subject exposed to a poison is provided which includes administering a pharmaceutical composition including a therapeutically effective amount of a chloride current modulator, an exogenous oxygen carrier or a combination of a chloride current modulator and an exogenous oxygen carrier to an individual subject who has been exposed to a poison. The therapeutically effective amount of the chloride current modulator, an exogenous oxygen carrier or combination thereof reduces a symptom or sign of a poison-caused pathology, thereby treating the poison-caused pathology. In a preferred embodiment, the chloride current modulator is an inhibitor of an ICl, swell chloride current.

In one embodiment, an inventive method further includes administering a therapeutic agent to inhibit poison-caused pathology. Illustrative examples of therapeutic agents include an ion current modulator, an antiarrhythmic drug, a toxic agent-clearing agent, an inhibitor of protein kinase C, a modulator of a mitochondrial membrane moiety, and a combination thereof.

Poisons which cause pathology for which methods and compositions of the present invention are provided include a toxic agent, a chemical asphyxiant, and combinations thereof. Illustrative examples of such toxic agents include a cyanide, an azide, carbon monoxide, and a combination of these. Illustrative examples of chemical asphyxiants include a toxic agent, hydrogen sulfide, nitrogen oxide, chlorine and a combination thereof. In one embodiment, the poison-caused pathology is a cardiac abnormality such as ventricular fibrillation or cardiogenic shock.

A composition according to the present invention is provided which includes at least one of the following: a chloride current modulator or an exogenous oxygen carrier. Such a composition may further include a combination of a chloride current modulator and an exogenous oxygen carrier. In one embodiment, an inventive composition preferably further includes a therapeutic agent to inhibit poison-caused pathology. In a further preferred embodiment, the chloride current modulator is an inhibitor of ICl, swell.

Exemplary exogenous oxygen carriers include hemoglobin-based oxygen carriers and fluorocarbon-based carriers. In one embodiment, an exogenous oxygen carrier includes a fluorocarbon oxygen carrier dispersed in an aqueous phase and a therapeutic agent is present in the aqueous phase.

A method of treating a toxic agent-caused cardiac abnormality in an individual subject is described which includes administering a pharmaceutical composition including a therapeutically effective amount of a chloride current modulator to an individual subject having a cardiac abnormality caused by poisoning with a toxic agent. The chloride current modulator is effective to modulate a chloride conductance and thereby reduce a symptom or sign of a toxic agent-caused cardiac abnormality, thus treating the toxic agent-caused cardiac abnormality. In a preferred option, the chloride current modulator is a modulator of a chloride current activated in response to changes in cell volume, the current known as I_{Cl,swell}. Modulators of chloride currents include: a disulfonic stilbene; an arylaminobenzoate; a fenamate; an anthracene carboxylate; an indanylalkanoic acid; clofibric acid; a clofibric acid derivative; a sulfonylurea; a calixarene; suramin; and tamoxifen. Further preferred are modulators of I_{Cl,swell} such as 4,4′-diisothiocyanostilbene-2,2

′-disulfonic acid (DIDS); 4,4′-dinitrostilbene-2,2′-disulfonic acid (DNDS); 4-acetamido-4′-isothiocyanostilbene-2,2′-disulfonic acid (SITS); tamoxifen; 5-nitro-2-(3-phenylpropylamino) benzoic acid (NPPB); niflumic acid (NFA); flufenamic acid; anthracene-9-carboxylate (9AC); diphenylaminecarboxylate (DPC); 2-(p-chlorophenoxy)propionic acid (CPP); and indanyloxyacetic acid (IAA-94). Mixtures of these modulators may also be administered.

Optionally, an inventive method further includes administering a therapeutic agent to inhibit toxic agent-caused distortion of the action potential of myocytes, support restoration of usual intracellular ionic concentrations, and support an increase in cardiac contractility. Such therapeutic agents include a second current modulator; an antiarrhythmic drug; a modulator of a mitochondrial membrane ion channel, ion pump and/or ion exchanger; an inhibitor of protein kinase C; a toxic agent-clearing agent; a cyanide-clearing agent; and combinations thereof.

A composition according to the invention is described which includes a chloride current modulator and a therapeutic agent to inhibit toxic agent-caused distortion of the action potential of myocytes, support restoration of usual intracellular ionic concentrations, and support an increase in cardiac contractility. In a preferred option, the chloride current modulator is a modulator of I_{Cl,swell}. Further optionally, the therapeutic agent included in an inventive composition is selected from the group consisting of: a second current modulator, an antiarrhythmic drug, a toxic agent-clearing agent; a cyanide-clearing agent, an inhibitor of protein kinase C, a modulator of a mitochondrial membrane moiety, and a combination thereof.

In a further embodiment, a method of providing oxygen to an individual subject having chemical asphyxiant-caused oxygen deprivation is provided which includes administering a therapeutically effective amount of an exogenous oxygen carrier to the individual subject.

Optionally such a method includes confirming exposure to a chemical asphyxiant by the individual subject. Also optionally included is administering a therapeutic agent to inhibit chemical asphyxiant-caused pathology.

Compositions according to an embodiment of the present invention are provided which include a therapeutically effective amount of an exogenous oxygen carrier and a therapeutically effective amount of a therapeutic agent to inhibit chemical asphyxiant-caused pathology. Such therapeutic agents include a cell membrane ion current modulator, an antiarrhythmic drug, a chemical asphyxiant-clearing agent, an inhibitor of protein kinase C, a modulator of a mitochondrial membrane moiety, and a combination thereof.

In one embodiment, an inventive composition preferably includes an emulsion having a therapeutically effective amount of a hydrophobic exogenous oxygen carrier, such as a fluorocarbon oxygen carrier, dispersed in an aqueous phase and a therapeutic agent present in the aqueous phase.

An embodiment of an inventive method of treating chemical asphyxiant-caused oxygen deprivation in an individual subject includes detecting a symptom or sign of chemical asphyxiant-caused oxygen deprivation in an individual subject and administering a therapeutically effective amount of an exogenous oxygen carrier to the individual subject, thereby causing an amelioration of the symptom or sign of chemical asphyxiant-caused oxygen deprivation and treating the chemical asphyxiant-caused oxygen deprivation. A further embodiment includes administering a therapeutic agent to inhibit chemical asphyxiant-caused pathology.

### BRIEF DESCRIPTION OF THE DRAWINGS

FIG. 1 shows a simulated electrocardiogram (ECG or EKG) illustrating a control trace, A, representative of normal cardiac activity, and a trace B illustrating differences due to cyanide; and

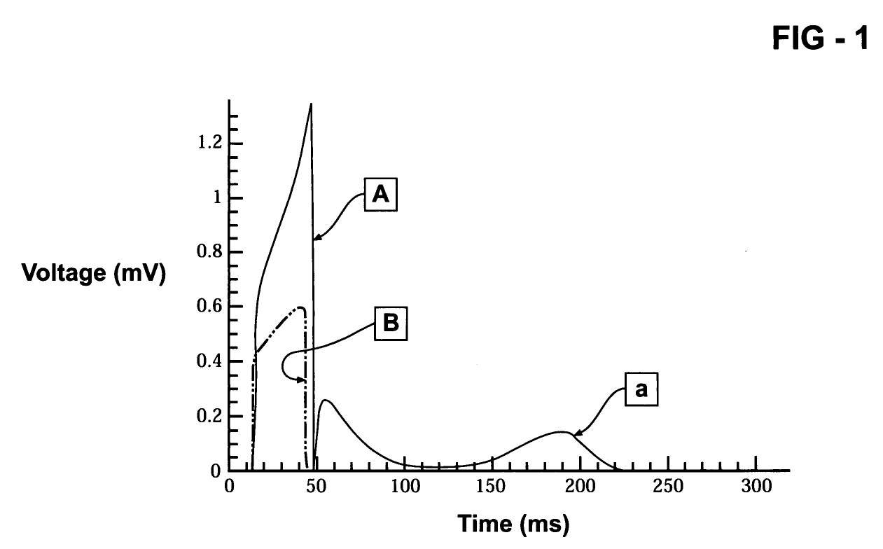

FIG. 2 is a graph showing simulated action potentials and changes thereof in various states relating to cyanide poisoning.

### DETAILED DESCRIPTION OF THE PREFERRED EMBODIMENTS

A method of treating a poison-caused pathology in an individual subject exposed to a poison is provided which includes administering a pharmaceutical composition including a therapeutically effective amount of a chloride current modulator, an exogenous oxygen carrier or a combination of a chloride current modulator and an exogenous oxygen carrier to an individual subject who has been exposed to a poison. The therapeutically effective amount of the chloride current modulator, an exogenous oxygen carrier or combination thereof reduces a symptom or sign of a poison-caused pathology, thereby treating the poison-caused pathology. In one embodiment, the poison-caused pathology is a cardiac abnormality such as ventricular fibrillation or cardiogenic shock.

Poisons which cause pathology for which methods and compositions of the present invention are provided include a toxic agent, a chemical asphyxiant, and combinations thereof. Illustrative examples of such toxic agents include a cyanide, an azide, carbon monoxide, and a combination of these. Illustrative examples of chemical asphyxiants include a toxic agent, hydrogen sulfide, nitrogen oxide, chlorine and a combination thereof.

A method of treating a toxic agent-caused cardiac abnormality in an individual subject is provided which includes administering a pharmaceutical composition containing a therapeutically effective amount of an ion channel modulator to an individual subject having a cardiac abnormality caused by poisoning with a toxic agent. The ion channel modulator is effective to modulate an ion channel conductance, thereby reducing a symptom or sign of a toxic agent-caused cardiac abnormality and treating the toxic agent-caused cardiac abnormality.

In one embodiment, a toxic agent that causes pathology for which compositions and methods of treatment are provided is a toxic agent which causes swelling of cardiac cells resulting in activation of a usually inactive membrane current, particularly a chloride current. Further, such a toxic agent for which compositions and methods of treatment are provided is a metabolic inhibitor, such as an inhibitor of oxidative phosphorylation, such that inhibition of metabolism contributes to cell swelling. A preferred type of toxic agent for which compositions and methods of treatment are provided is a metabolic inhibitor which inhibits a metal containing component of the electron transport chain, such as cytochrome c oxidase. Illustrative examples of toxic agents which cause cardiac pathology for which compositions and methods of treatment are provided include cyanides. In a preferred embodiment, cyanide toxic agents are those cyanides which release a cyanide ion (CN—) and/or form HCN in aqueous solution such as a physiological fluid of a mammal. Such cyanides include hydrogen cyanide itself as well as alkalai salts such as NaCN, KCN, Ca(CN)2 and Hg(CN)2. Additional cyanide toxic agents are described herein. Azides are a further illustrative example of toxic agents, including organic azides, metallic azides and inorganic azides. In a preferred embodiment, azide toxic agents are those azides which release an azide ion in aqueous solution such as a physiological fluid of a mammal. In a particular embodiment sodium azide is a toxic agent for which compositions and methods of treatment are provided. Carbon monoxide is a further example of such a toxic agent which binds and inhibits a metal-containing component of the electron transport chain.

A therapeutically effective amount is that amount which decreases a symptom or sign of toxic agent-caused cardiac abnormalities. Exemplary symptoms and signs include arrhythmia, fibrillation, abnormal ECG readout such as changes in the P-wave, the depolarization of the atria, and prominent modulation of the T-wave, rising resting potential, Brugada-like symptoms, an initial bradycardia, Torsade de Pointes and tachycardia. Such signs and symptoms are monitored by methods typically used to monitor cardiac parameters, such as ECG. Further symptoms and signs are described herein. In some cases, symptoms and signs associated with poisoning are not specifically associated with toxic agent exposure. In such cases, poisoning may be confirmed by tests for presence of the toxic agent, a metabolite of the toxic agent, a degradation product of the toxic agent and/or a personal history indicative of likelihood of exposure to the toxic agent.

An embodiment of an inventive method includes treatment of disorders associated with toxic agent toxicity, particularly cardiac abnormalities such as heart failure caused by ventricular fibrillation due to toxic agent-caused toxicity and cardiogenic shock due to toxic agent-caused toxicity.

One characteristic of toxic agent poisoning is distortion of ion gradients across excitable membrane cell membranes, particularly cardiac cell membranes. Thus, an inventive method includes administering a compound to modulate toxic agent-caused activation and/or inhibition of one or more cardiac cell transmembrane ion currents, so as to inhibit toxic agent-caused distortion of the action potential of myocytes, support restoration of usual intracellular ionic concentrations, and support an increase in cardiac contractility.

The present invention provides methods for modulation of selected membrane currents to aid in inhibiting toxic agent-caused distortion of the action potential of myocytes, support restoration of usual intracellular ionic concentrations, and support an increase in cardiac contractility. It has been found by the present inventors that modeling inhibition of a toxic agent-caused activation of a membrane current which is typically swelling-activated has such beneficial effects on modeled cell parameters.

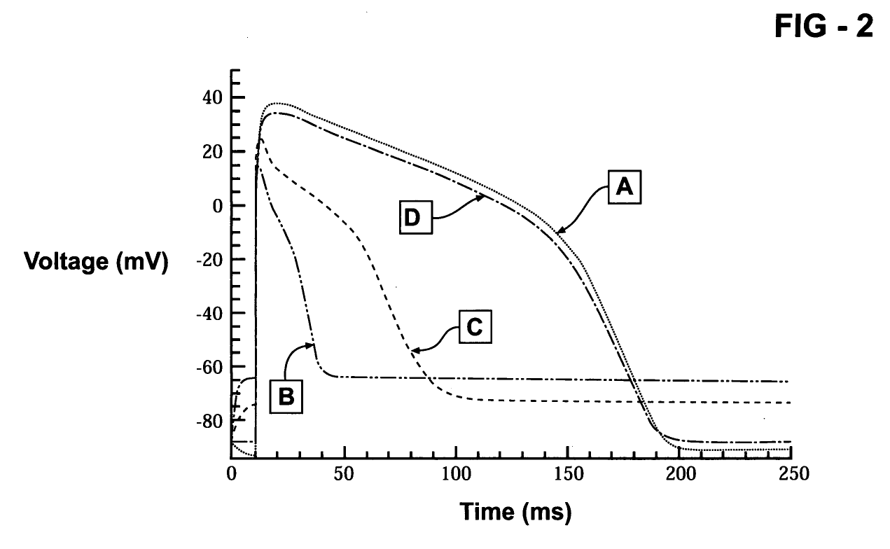

In a particular example, modeled inhibition of a transmembrane chloride current activated in a cyanide exposed cell or tissue aids in reestablishing characteristics of normal cell and tissue functionality, for instance by inhibiting cyanide-caused distortion of the action potential of myocytes, supporting restoration of usual intracellular ionic concentrations, and supporting an increase in cardiac contractility. FIG. 2 illustrates a model of cyanide poisoning of cardiac cells and the effect of terminating the cell membrane chloride current. A normal action potential typical of cardiac tissue unaffected by cyanide is shown in the rightmost trace (A) representing a control. The leftmost trace (B) shows a model of an action potential of a cyanide affected cardiac ventricular cell. Note the shortened cycle length, the raised equilibrium potential and the reduced amplitude. Restoring the usual potassium ion concentrations across the cell membrane helps, as shown in (C), but the cycle length, a measure of the tachycardia, is still less than half of the control condition. Antagonizing the cyanide-caused chloride membrane current, as shown in the trace (D) restores the normal action potential, indicating restored membrane potential and contractility.

Thus, compounds administered in a method according to the invention include those that inhibit usually inactive membrane currents activated following exposure of tissue to a toxic agent.

In one embodiment, an inventive method of treating toxic agent poisoning in an individual subject includes administering a pharmaceutical composition which contains a therapeutically effective amount of a chloride channel modulator to an individual subject in need thereof.

In a particular embodiment, the chloride channel modulator is an antagonist of a chloride current activated in a cell exposed to a toxic agent. In a preferred embodiment, the chloride current modulator is a modulator of a chloride current known as “I_{Cl,swell}.” I_{Cl,swell} is a cell volume regulated chloride current present in cardiac cells. See, for example, Hume, J. R. et al., Physiol. Reviews, 80:31-81, 2000 and Lang, F. et al., Physiol. Reviews, 78:247-306, 1998.

Chloride current antagonists illustratively include disulfonic stilbenes, arylaminobenzoates, fenamates, anthracene carboxylates, indanylalkanoic acids, clofibric acid, clofibric acid derivatives, sulfonylureas, calixarenes, suramin, and tamoxifen.

Particular chloride conductance modulators administered in an embodiment of an inventive method include inhibitors of an “I_{Cl,swell}” chloride current. For example, a preferred disulfonic stilbene inhibitor of an “I_{Cl,swell}” chloride current included in a method and composition according to the invention is 4,4′-diisothiocyanostilbene-2,2′-disulfonic acid (DIDS). In another embodiment, disulfonic stilbenes included in a method and composition according to the invention illustratively include 4,4′-dinitrostilbene-2,2′-disulfonic acid (DNDS) and 4-acetamido-4′-isothiocyanostilbene-2,2′-disulfonic acid (SITS).

In another embodiment, an “I_{Cl,swell}” chloride current inhibitor included in an inventive method and composition is tamoxifen. In further embodiments, an “I_{Cl,swell}” chloride current inhibitor included in an inventive method and composition is illustratively 5-nitro-2-(3-phenylpropylamino) benzoic acid (NPPB); niflumic acid (NFA); flufenamic acid; anthracene-9-carboxylate (9AC); diphenylaminecarboxylate (DPC); 2-(p-chlorophenoxy)propionic acid (CPP—a clofibric acid derivative); and indanyloxyacetic acid (IAA-94).

An inhibitor of “I_{Cl,swell}” chloride current may be identified using methods known in the art. For example, one or more cells placed in a recording chamber may be exposed to a stimulus causing a change in cell volume. A chloride current activated in response to the change in cell volume may be detected by methods such as whole cell or patch clamp techniques. An inhibitor of “I_{Cl,swell}” chloride current may be identified as an agent that causes a specific decrease in “I_{Cl,swell}” chloride current.

A chloride conductance inhibitor included in embodiments of a composition and method according to the invention preferably produces at least about a 50% decrease in a toxic agent exposure-caused membrane chloride current. Further, a chloride conductance inhibitor included in embodiments of a composition and method according to the invention preferably induces at least about a 50% increase in cardiac action potential duration in a toxic agent-poisoned substrate, that is, the tissue being acted on.

One consequence of toxic agent poisoning is deleterious accumulation of intracellular calcium. A chloride conductance inhibitor included in a composition and method according to the invention causes at least about a 1-12% decrease in intracellular calcium concentration and preferably induces at least about a 2-6% decrease in intracellular calcium concentration. Such a decrease in intracellular calcium concentration may be measured in a standard in vitro calcium sensitizing assay, such as that detailed in M. Endoh, Mechanism of action of novel cardiotonic agents, J. Cardiovascular Pharmacology, 2002, 40:323-338.

Cyanide poisoning is a toxicity caused by multiple effects of the cyanide compounds on metabolic processes. Thus, cardiac cells are affected by cyanide via several pathways and cyanide-related cardiac pathology appears to be the result of such multiple effects. Some of the toxic effects of cyanide occur by mechanisms such as the affinity of cyanide compounds for metal ions, such as those found in metal-containing enzymes such as cytochrome c oxidase and catalase. In particular, an effect of cyanide binding to cytochrome c oxidase is inhibition of cellular respiration, leading to a reduction in cardiac muscle contractility. (Baskin et al., 1987, Cardiac Effects of Cyanide, Clinical Experimental Toxicology of Cyanides, p. 138-155, Ballantyne, B. and Marrs, T. C., eds., Wright Bristol). Cyanide poisoning may also produce constriction of particular blood vessels and particularly the pulmonary arterial and/or coronary vessels resulting in decreased cardiac output (Vick and Froelich, 1985, Studies of Cyanide Poisoning. Arch. Int. Pharmacodyn, 273:314-322.) In addition, cyanide compounds are believed to cause a release of biogenic amines which may also have toxic effects (Burrows, G. E. and Way, J. L., 1976, Antagonism of Cyanide Toxicity by Phenoxybenzamine, Fed. Proc., 35:533). For example, catecholamine release occurs that activates both α: and β adrenoreceptors. This results in increased oxygen consumption and higher intracellular loading in cardiac cells.

Azide is a metabolic inhibitor which inhibits cytochrome c oxidase and thus inhibits oxidative phosphorylation by binding to cytochrome a3 in its ferric (oxidized) form thereby blocking electron transport and ATP synthesis. Due to the large demand for oxygen in the heart as well as the brain, the effect of azides when in contact with these organs is critical. See, for example, Shearer J et al. PNAS 2003;100:3671-3676, and Schwoebel, ED et al. J Cell Biol 2002;157:963-974. Further, sodium azide is a mitochondrial uncoupler and directly activates KATP channels, see, for example, Harvey, J. et al British J Pharmacology 1999;126:51-60, and Trapp, S. et al. ibid 2000;131:1105-1112. Currently, no specific antidote is believed to exist for sodium azide poisoning.

A conductance modulator is optionally delivered in conjunction with a therapeutic agent in addition to a conductance modulator in one embodiment. A therapeutic agent may be included in a pharmaceutical composition with a conductance modulator and/or administered separately.

A composition including a chloride current modulator, particularly an inhibitor of I_{Cl,swell} and a therapeutic agent, is administered to inhibit toxic agent-caused distortion of the action potential of myocytes, support restoration of usual intracellular ionic concentrations, and support an increase in cardiac contractility. Exemplary therapeutic agents illustratively include a second current modulator, an antiarrhythmic drug, a toxic agent-clearing agent, a cyanide-clearing agent, an inhibitor of protein kinase C, a modulator of a mitochondrial membrane moiety, and a combination thereof. Combinations of a chloride current modulator, particularly an inhibitor of I_{Cl,swell} and a therapeutic agent may have a synergistic effect in inhibiting toxic agent-caused distortion of the action potential of myocytes, supporting restoration of usual intracellular ionic concentrations, and supporting an increase in cardiac contractility. In particular, lesser amounts of a chloride current modulator may be necessary to achieve reduction in a symptom or sign of cardiac abnormality when the modulator is included in a composition with a therapeutic agent. Such a composition may therefore provide benefits of treatment of toxic agent-caused cardiac abnormality as well as cost savings and reduction in side effects. Further, lesser amounts of a therapeutic agent may be administered when included in an inventive composition. A therapeutically effective amount of a therapeutic agent administered to inhibit toxic agent-caused distortion of the action potential of myocytes, support restoration of usual intracellular ionic concentrations, and support an increase in cardiac contractility is an amount which is effective to achieve a desired result indicated by the choice of the agent.

An exemplary therapeutic agent suitable in this regard is a toxic agent-clearing agent. The term “toxic agent-clearing agent” as used herein is intended to mean a compound having a beneficial effect on a toxic agent-poisoned individual, particularly an effect of inhibiting the action of a toxic agent in the affected individual such as by stimulating removal, sequestration, or metabolism of the toxic agent such that the toxic effects are inhibited.

A particular exemplary toxic agent-clearing agent is a cyanide-clearing agent. A cyanide clearing agent illustratively includes a sulfur donor for a sulfur transferase. In one embodiment, a sulfur donor included in an inventive composition is a sulfur donor for an endogenous sulfur transferase, such as rhodanese. A cyanide clearing agent may further be a compound reacting with hemoglobin to form a compound competitive with cytochrome c oxidase for cyanide. In particular, a cyanide clearing agent may react with hemoglobin to form methemoglobin. Examples of compounds reacting with hemoglobin to form methemoglobin include nitrites such as sodium nitrite or amyl nitrite. A further example of a compound reacting with hemoglobin to form methemoglobin is 4-dimethylaminophenol (4-DMAP). A further example of a compound reacting with hemoglobin to form a compound competitive with cytochrome c oxidase for cyanide is sulfur hemoglobin. Additional cyanide clearing agents include cyanide binding agents illustratively including hydroxocobalamin and dicobalt edetate.

Exemplary doses for a cyanide clearing agent include, each independently or in combination, 50 ml of 25% sodium thiosulfate solution (12.5 g) i.v. for 10 minutes, 20 ml of 1.5% dicobalt edetate solution (300 mg) i.v. for 1 minute, 10 ml of 40% hydroxocobalamin solution (4 g) i.v. for 20 minutes, 10 ml of 3% sodium nitrite solution (300 mg) i.v. for 5-20 minutes; and 5 ml of 5% 4-DMAP solution (250 mg or 3-4 mg/kg) i.v. for 1 minute. One of skill in the art will know how to adjust such doses in order to meet the demands of a particular therapeutic situation.

In a further example of a therapeutic agent, a second conductance modulator is optionally administered in order to inhibit toxic agent-caused distortion of the action potential of myocytes, support restoration of usual intracellular ionic concentrations, and support an increase in cardiac contractility. For example, more than one modulator of a chloride current may be administered. The multiple chloride current modulators optionally modulate the same or different chloride channels. In another example, such a compound is a sodium channel opener such as aconitine, and/or such as veratridine which slows the sodium current inactivation, (Wang, G K and Wang S Y, Veratridine block of rat skeletal muscle Nav1.4 sodium channels in the inner vestibule, J Physiol., 2003, 548:667-675.), or a calcium channel opener such as BAY K 8644 and/or flecanide.

Another example of a therapeutic agent is an anti-arrhythmic drug administered in order to further support inhibition of toxic agent-caused distortion of the action potential of myocytes, restoration of usual intracellular ionic concentrations, and an increase in cardiac contractility. Antiarrhythmic drugs illustratively include class I antiarrhythmics which are sodium channel blockers such as class IA antiarrhythmics illustratively including quinidine (Quinidex), procainamide (Pronestyl) and disopyramide (Norpace); class IB antiarrhythmics illustratively including lidocaine (Xylocaine), tocainide (Tonocard), and mexiletine (Mexitil); class IC antiarrhythmics illustratively including encainide (Enkaid), and flecainide (Tambocor); class II antiarrhythmics which are beta blockers illustratively including propranolol (Inderal), acebutolol (Sectral), esmolol (Brevibloc), metoprolol, and atenolol; class III antiarrhythmics which are potassium channel blockers illustratively including sotalol (Betapace), dofetilide (Tikoyn) and amiodarone (Cordarone) and class IV antiarrhythmics which are calcium channel blockers illustratively including verapamil (Calan, Isoptin), diltiazem (Cardizem) and mebefradil (Posicor). In addition, some antiarrhythmics such as alinidine may act as chloride channel blockers, see Millar J S and Williams E M., Pacemaker selectivity: influence on rabbit atria of ionic environment and of alinidine, a possible anion antagonist. Cardiovasc. Res. 1981, 15(6):335-50. Digoxin is a further antiarrhythmic optionally administered. Dosage of such agents is known in the art and is illustrated in standard texts such as R. N. Fogoros, Antiarrhythmic Drugs: A Practical Guide, Blackwell Publishers, 1997, and Mosby's Drug Consult, 2005, Mosby Inc., Elsevier, St. Louis Mo., ISBN 0-323-03393-8. For example, quinidine gluconate is typically administered in doses ranging from about 5-10 mg/kg (Mosby's Drug Consult p. II-2466-2475); procainamide is generally given in amounts of up to 50 mg/kg/day (Mosby's Drug Consult p. II-2409-2412); disopyramide is administered in amounts ranging from about 400-1200 mg/day (Mosby's Drug Consult p. II-852-855); lidocaine is generally given in amounts in the range of 50-100 mg administered intravenously (Mosby's Drug Consult p. II-1744-1751); tocainide is usually given in amounts ranging from 1200-1800 mg/day (Mosby's Drug Consult p. II-2833); mexiletine is generally given in amounts of about 600-1200 mg/day (Mosby's Drug Consult p. II-1951-1953); flecainide is usually administered in amount ranging from about 100-300 mg/day (Mosby's Drug Consult p. II-1189-1190); propranolol is typically given in amounts in the range of about 80-640 mg/day (Mosby's Drug Consult p. II-2445-2451); acebutolol is generally given in amounts in the range of about 200-1200 mg/day (Mosby's Drug Consult p. II-13-14); esmolol is typically administered in amounts ranging from about 50-500 micrograms/kg/min (Mosby's Drug Consult p. II-1019-1022); sotalol is generally given in amounts ranging from 160-640 mg/day (Mosby's Drug Consult p. II-2642-2650); dofetilide is generally given in amounts ranging from 250-1000 micrograms/day (Mosby's Drug Consult p. II-872-876); amidarone is usually administered in an amount in the range of about 400-1600 mg/day in oral form (Mosby's Drug Consult p. II-121-130); verapamil is typically given in an amount in the range of about 120-480 mg/day (Mosby's Drug Consult p. II-2977-2983); digoxin is administered as described in Mosby's Drug Consult p. II-810-819; and diltiazem is generally administered in amounts in the range of about 180-360 mg/day in oral formulation (Mosby's Drug Consult p. II-827-839).

In one embodiment, a therapeutic agent is administered which addresses effects of toxic agent poisoning on mitochondrial function. As noted above, one of the effects of toxic agent poisoning is inhibition of oxygen metabolism. Consequences of such inhibition include distortion of usual ion concentrations across the mitochondrial membrane. For example, a particular problem is accumulation of calcium in mitochondria, due in part to insufficient production of ATP necessary to sustain activity of mechanisms for maintenance of normal calcium concentrations, such as the Na⁺-Ca⁺⁺ exchanger. Further, sodium and potassium concentrations in mitochondria are distorted due, at least in part, to the decreased production of ATP and consequent inhibition of a Na⁺-K⁺ pump. A further deleterious effect of cyanide poisoning on cells and mitochondria is increased generation of reactive oxygen species.

A therapeutic agent which addresses effects of toxic agent poisoning on mitochondrial function includes an agonist or antagonist of a mitochondrial membrane channel ion exchanger and/or pump effective to normalize distorted ion concentrations across the mitochondrial membrane. The mitochondrial membrane moiety may be selected from the group consisting of: an ion channel, an ion pump, an ion exchanger. Such compounds may be included to improve mitochondrial energy production, dampen mitochondrial calcium accumulation and reactive oxygen species production. For example, since calcium concentrations inside the mitochondria are typically increased following cyanide exposure, an agent may be administered which is effective to activate or enhance an outward movement of calcium from a mitochondrion. Exemplary agents effective to stimulate calcium efflux include menadione (Henry T R et al., J. Toxicol. Environ. Health, 45(4):489-504, 1995).

In other examples, an agonist or antagonist of a mitochondrial membrane channel and/or pump stimulates activity of a mitochondrial membrane potassium channel, such as mitoKATP and/or mitoKCa. A particular compound which stimulates mitoKATP is diazoxide (Proglycen).

Optionally further included is administration of a therapeutic agent which is a protein kinase C (PKC) inhibitor. In the presence of cyanide, rising inorganic phosphate (Pi) and declining phosphocreatine (PCr) are present in cells, affecting the electrophysiology of the tissue. An exemplary PKC inhibitor which may be used is H-7 (1-(5-isoquinolinesulfonyl)-2-methylpiperazine, an isoquinolinesulfonamide), a pharmacological inhibitor of PKC which prevents and/or inhibits the changes in the PCr and Pi.

An embodiment of the present invention addresses the effects of toxic agents which limit oxygen availability in cells and tissues affected by the toxic agent, causing oxygen deprivation. A toxic agent which causes oxygen deprivation is a chemical asphyxiant, that is, a compound that interferes with the distribution, uptake, and utilization of oxygen in an individual subject. In general, chemical asphyxiant toxic agents include those toxic agents described herein which cause cardiac abnormalities. Optionally, chemical asphyxiant toxic agents further include toxic agents which interfere with the distribution, uptake, and utilization of oxygen in an individual subject by various other mechanisms, such as damage to lung tissue by the chemical asphyxiant toxic agent chlorine. Illustrative examples of chemical asphyxiants are carbon monoxide, hydrogen sulfide, and nitrogen oxides. Of particular interest are cyanide asphyxiants including hydrogen cyanide and other cyanide toxic agents as described herein.

Signs and symptoms of oxygen deprivation caused by such a chemical asphyxiant include headache, flushing, diaphoresis, tachycardia, and hypertension, as well as loss of consciousness, convulsions, and coma. Signs and symptoms of oxygen deprivation caused by cyanide are described herein.

As noted above, some of the toxic effects of cyanide are a result of the affinity of cyanide compounds for metal ions, such as those found in metal-containing enzymes, including cytochrome c oxidase. An effect of cyanide binding to cytochrome c oxidase is inhibition of cellular respiration. The poison also attaches to hemoglobin, preventing the delivery of oxygen required by cells, as well as impeding the removal of waste products such as CO₂.

Thus, in an individual affected by a chemical asphyxiant which inhibits normal oxygen transport and/or cellular respiration, it is important to provide oxygen to cells and thereby aid in maintaining the energy supply needed for cell function.

A conventional treatment for an individual affected by cyanide poisoning and other forms of chemical asphyxiant poisoning is administration of oxygen. However, where few endogenous hemoglobin molecules are available for binding and delivery of oxygen, administration of oxygen alone may be a less than optimal treatment, since little or no oxygen carrier is available for transport to cells. It is known from deep-sea diving research that hyperbaric oxygen increases the serum dissolved oxygen to levels adequate for life. Further, hyperbaric oxygen increases cytochrome oxidase activity. An adverse effect though is that this decreases the oxygen carrying capacity of the blood resulting in tissue hypoxia. At sea level, under normal circumstances, the blood plasma oxygen concentration is 0.3 mL/dL. At normobaric pressures, 100% oxygen increases the oxygen concentration of the blood five-fold, at 3 atm this rises to twenty-fold. At this level, the oxygen requirement of the organs is met without the oxygen carried by the hemoglobin. However, hyperbaric oxygen chambers are not available in first responder situations. Further, the effects of some chemical asphyxiants, such as cyanide, on cellular respiration and oxygen transport are virtually immediate following exposure to the chemical asphyxiant, making restoration of oxygen delivery to the cells a priority. Since removal, sequestration and/or inactivation of chemical asphyxiants in the affected organism can take time, an oxygen carrier other than the endogenous hemoglobin is needed to temporarily aid in oxygen delivery as well as in removal of waste products, such as carbon dioxide.

Currently, methods and compositions for restoring oxygen delivery to cells and tissues in need of oxygen are needed for treatment of an poisoned individual. Such methods and compositions are provided by the present invention.

Inventive methods are provided which include administering an exogenous gas carrier to an individual for supplying oxygen to tissues and cells in need thereof due to chemical asphyxiant exposure. An exogenous oxygen carrier is administered to a chemical asphyxiant poisoned individual in order to carry oxygen to tissues and cells where a portion or all of the usual endogenous oxygen carrier, hemoglobin, is unavailable for the task due to the effects of the chemical asphyxiant.

In a further embodiment, an exogenous gas carrier is administered in conjunction with a treatment and/or therapeutic agent aimed at clearing the chemical asphyxiant and/or ameliorating its toxic effects in an affected individual, thus treating a chemical asphyxiant-caused pathology.

A method of providing oxygen to an individual subject having chemical asphyxiant-caused oxygen deprivation according to an embodiment of the invention includes intravenously administering a therapeutically effective amount of an exogenous oxygen carrier to the individual subject.

Exogenous oxygen carriers include hemoglobin-based oxygen carriers and fluorocarbon-based oxygen carriers. Hemoglobin-based exogenous oxygen carriers include natural non-human animal hemoglobins, modified or synthetic human hemoglobin, modified or synthetic non-human animal hemoglobins, and protein conjugate oxygen carriers, such as albumin-heme. Fluorocarbon-based exogenous oxygen carriers include perfluorinated carbon compounds and fluorine substituted hydrocarbons.

Exemplary hemoglobin-based exogenous oxygen carriers include those described in U.S. Pat. Nos.: 6,518,010; 6,506,725; 6,271,351; 6,150,507; 5,955,581; 5,952,470; 5,905,141; 5,895,810; 5,854,209; 5,840,852; 5,753,616; 5,691,453; 5,691,452; 5,618,919; 5,296,465; and 5,084,558.

A further embodiment of this invention uses a modified hemoglobin-based exogenous oxygen carrier. These illustratively include a polymerized human or animal hemoglobin and a recombinant human or animal hemoglobin. Examples of commercial products including such materials include POLYHEME and HEMOPURE. An advantage of some animal hemoglobins, such as bovine hemoglobin, is non-recognition by the human immune system as foreign.

A further exogenous oxygen carrier based on modified hemoglobin is a polyethylene glycol-modified hemoglobin such as described in U.S. Pat. Nos. 6,432,918; 6,054,427; 5,985,825; and 5,814,601.

Also suitable is the “albumin-heme” class of exogenous oxygen-carrying plasma proteins. These incorporate synthetic heme into recombinant human serum albumin (rHSA) that can reversibly bind and release oxygen similar to hemoglobin. An added advantage is that synthetic preparations are free of pathogens which may be transferred from animal products.

Fluorocarbon exogenous oxygen carriers, including perfluorinated carbon compounds and fluorine substituted hydrocarbons are suitable for use in an inventive composition and method.

Fluorocarbon exogenous oxygen carriers have high oxygen dissolving characteristics, low water solubility and are inert, favoring their use in oxygen supplemental situations. Solubility of oxygen in fluorocarbon oxygen carriers is directly proportional to the partial pressure of the gas.

Fluorocarbon exogenous oxygen carriers have a half-life of hours to days following intravenous administration to an organism. Inert fluorocarbon exogenous oxygen carriers are not metabolized in vivo and are typically eliminated unaltered through the lungs.

Suitable fluorocarbon oxygen carriers dissolve other gases in addition to oxygen. Thus, fluorocarbon exogenous oxygen carriers have the advantage of being capable of oxygen delivery and, additionally, carrying CO₂ for removal from the body.

A fluorocarbon exogenous oxygen carrier is a linear, branched, cyclic, saturated or unsaturated fluorinated carbon compound. Particularly preferred are perfluorocarbon compounds. Characteristics of fluorocarbon oxygen carriers suitable for use in vivo are described herein, in Reiss, J. G., Artif. Cells, Blood Subst. Biotech., 33:47-63, 2005, and in patents cited herein.

In one embodiment, perfluorocarbon exogenous oxygen carriers are perfluorocarbons having the chemical formulas CₙF₂ₙ₊ₓ, CₙF₂ₙ, CₙF₂ₙ₋ₓ and CₙFₙ, where n is an integer in the range from 1-20, inclusive, and where x is an integer in the range from 1-10. Suitable compounds have a molecular weight in the range of about 200-800. Optionally, and preferably, the perfluorocarbon exogenous oxygen carrier is a liquid at temperatures in the range of room temperature and normal human body temperature, about 20-37° C.

Further suitable fluorocarbon exogenous oxygen carriers include those having the chemical formulas CₙR₂ₙ₊ₓ, CₙR₂ₙ, CₙR₂ₙ₋ₓ and CₙRₙ, where n is an integer in the range from 1-20, inclusive, where x is an integer in the range from 1-10, where each R is independently selected from halogen, H, O, OH, S, N, NH₂, and NH₃, with the proviso that at least one and preferably at least two of the R substituents are F. Suitable compounds have a molecular weight in the range of about 200-800. Optionally, and preferably, the fluorocarbon exogenous oxygen carrier is a liquid at temperatures in the range of room temperature and normal human body temperature, about 20-37° C.

Exemplary suitable fluorocarbon exogenous oxygen carriers include bis(F-alkyl) ethanes illustratively including C₄F₉CH═CHC₄F₉, i-C₃F₉CH═CHC₆F₁₃, and C₆F₁₃CH═CHC₆F₁₃; and cyclic perfluorocarbons, such as perfluorodecalin, perfluorinated adamantane, perfluorinate methyladamantane, and perfluorinated 1,3-dimethyladamantane.

Brominated fluorocarbons such as described in U.S. Pat. No. 3,975,512 may be used, as well as brominated perfluorocarbons, illustratively including 1-bromo-heptadecafluoro-octane, 1-bromo-pentadecafluoro-heptane, and 1-bromo-tridecafluoro-hexane.

Fluorinated and perfluorinated amines may be used, illustratively including F-tripropylamine and F-tri-butylamine, F-4-methyloctahydroquinolizine, F-n-methyl-decahydroisoquinoline, F-n-methyldecahydroquinoline, F-n-cyclohexylpyrrolidine and F-2-butyltetrahydrofuran.

Fluorinated and perfluorinated ethers and poly ethers may be used, illustratively including such compounds as (CF₃)₂CFO(CF₂CF₂)₂OCF(CF₃)₂,(CF₃)₂CFO, (CF₂CF₂)₃OCF(CF₃), (CF₃)CFO(CF₂CF₂)F, (CF₃)₂CFO(CF₂CF₂)₂F, and (C₆F₁₃)₂O.

Further illustrative fluorocarbon exogenous oxygen carriers are disclosed in U.S. Pat. Nos. 6,204,296; 5,344,393; 5,874,481; 5,635,538; 5,567,765; 6,537,246; 5,914,352; 4,868,318; 4,605,786; 5,403,575; 5,502,094; 5,785,950; 3,962,439; 4,423,077; 4,497,829; 5,434,191; 4,865,836; 5,451,205; and in other patents cited herein as exemplifying fluorocarbon emulsion compositions.

Perfluorocarbon oxygen carriers have been formulated for medical use as exemplified by FLUOSOL DA (Green Cross Corporation, Japan), which includes perfluorodecalin and perfluorotripropylamine, as well as OXYGENT (Alliance Pharmaceutical Corp., San Diego, USA), which includes perfluorooctyl bromide.

The FDA has approved the use of FLUOSOL DA, a perfluorocarbon oxygen carrier as a blood substitute during heart surgery. Also clinically used is OXYGENT, an agent that has completed Phase 3 clinical trial in Europe.

A therapeutically effective amount of an exogenous oxygen carrier is an amount which decreases a symptom or sign of toxic agent-caused oxygen deprivation. In general, the amount of a fluorocarbon oxygen carrier administered to a toxic agent poisoned individual is in the range of about 01 g-10 g/kg body weight of the individual.

In general, fluorocarbon and perfluorocarbon gas carriers are highly hydrophobic and are thus provided as emulsions, the making and composition of which are exemplified in references U.S. Pat. Nos. 6,528,545; 5,635,538; 4,895,876; 4,987,154; 5,171,755; 5,374,624; 5,514,720; 5,635,538; 5,914,352; other patents cited herein including fluorocarbon gas carrier compositions; and in Reiss, J. G., Artif. Cells, Blood Subst. Biotech., 33:47-63, 2005.

An exogenously supplied gas carrier is typically delivered in conjunction with a pharmaceutically acceptable vehicle included in an emulsion. A pharmaceutically acceptable vehicle is typically an aqueous vehicle which may include various components and excipients illustratively including a diluent, a buffering agent, an electrolyte, an emulsifier, an osmotic agent, and an antimicrobial.

As noted above, a therapeutic agent for treatment of chemical asphyxiant toxic agent-caused pathology associated with chemical asphyxiant toxic agent-caused oxygen deprivation is optionally administered according to an inventive method. Exemplary preferred therapeutic agents are selected from the group including a cell membrane current modulator, an antiarrhythmic drug, a chemical asphyxiant toxic agent clearing agent, an inhibitor of protein kinase C, a modulator of a mitochondrial membrane moiety. Combinations of such therapeutic agents are also contemplated.

A therapeutically effective amount of a therapeutic agent to inhibit chemical asphyxiant-caused pathology associated with oxygen deprivation is an amount which is effective to achieve a desired result indicated by the choice of the agent. For example, where a chemical asphyxiant toxic agent clearing agent is administered, a therapeutically effective amount of the agent is an amount effective to lessen the action of toxic agent in the affected individual such as by stimulating removal, sequestration, or metabolism of the toxic agent such that the toxic effects are inhibited. A preferred chemical asphyxiant toxic agent clearing agent is a cyanide clearing agent. In a further example, a current modulator is administered to aid in restoration of normal cell and tissue functionality, for instance by inhibiting cyanide-induced distortion of the action potential of myocytes, supporting restoration of usual intracellular ionic concentrations, and supporting an increase in cardiac contractility. An antiarrhythmic drug may be administered where a sign or symptom of toxic agent exposure is cardiac arrhythmia and to further support restoration of usual intracellular ionic concentrations. An inhibitor of protein kinase C and/or a modulator of a mitochondrial membrane moiety may be administered as required to prevent and/or inhibit the changes in the PCr and Pi and to inhibit distortion of usual ion concentrations across the mitochondrial membrane, respectively.

Further optionally included in an embodiment of an inventive method for providing oxygen to an individual having chemical asphyxiant-caused oxygen deprivation is confirmation of the affected individual's exposure to a chemical asphyxiant. Thus, exposure of the individual subject to a toxic agent may be confirmed in order to minimize treatment of individuals unaffected by a chemical asphyxiant. For instance, a history of the individual's recent activity is taken from the affected individual or from a bystander to assess the likelihood of chemical asphyxiant exposure. In some cases a quantitative or qualitative test is given to detect presence of a chemical asphyxiant or a metabolite of a chemical asphyxiant. For example, in the case of carbon monoxide poisoning, carboxyhemoglobin may be detected in a blood sample by spectrophotometric measurement using a blood gas analyzer. Further, exhaled carbon monoxide may be detected in exhaled gases. Optionally, cyanide exposure is confirmed by assay for cyanide, a cyanide metabolite, or a clinical and/or pathophysiological finding consistent with cyanide exposure.

Compositions according to an embodiment of the present invention are provided for supplying oxygen to an individual subject having toxic agent-caused oxygen deprivation which include a therapeutically effective amount of an exogenous oxygen carrier; and a therapeutically effective amount of a therapeutic agent to inhibit toxic agent-caused pathology. Exemplary preferred therapeutic agents include a current modulator, an antiarrhythmic drug, a chemical asphyxiant-clearing agent, an inhibitor of protein kinase C, a modulator of a mitochondrial membrane moiety, and a combination of two or more of these.

In one embodiment, an inventive composition is formulated as an emulsion including an effective amount of a fluorocarbon exogenous oxygen carrier dispersed in an aqueous phase. The composition further includes an effective amount of a therapeutic agent in the aqueous phase. Such an emulsion generally includes an emulsifying agent as well as one or more excipients illustratively including a diluent, a buffering agent, an electrolyte, an osmotic agent, and an antimicrobial.

Exemplary compositions according to the invention include an emulsion having an effective amount of an exogenous oxygen carrier dispersed in an aqueous phase and therapeutic agent such as a current modulator, an antiarrhythmic drug, a chemical asphyxiant-clearing agent, an inhibitor of protein kinase C, a modulator of a mitochondrial membrane moiety, or a combination of these present in the aqueous phase. Such a composition may be formed by, for instance, adding a therapeutic agent to an aqueous carrier, dissolving an exogenous oxygen carrier in a non-aqueous solvent and mixing the aqueous carrier/therapeutic agent with the exogenous oxygen carrier/non-aqueous solvent to form an emulsion. Such mixing may be by vigorous mechanical mixing and or sonication for instance. In general, the ratio of aqueous carrier/therapeutic agent to exogenous oxygen carrier/non-aqueous solvent is in the range of about 100:0.1-1:1. In a further embodiment the ratio is in the range of about 50:0.5-10:1.

Optionally, oxygen is dissolved in an exogenous oxygen carrier composition prior to administration to an individual in need thereof. Further optionally, oxygen is administered to the chemical asphyxiant-affected individual.

In an example of an inventive method and composition for providing oxygen to an individual having chemical asphyxiant-caused oxygen deprivation, various parameters are considered. An average adult has 5.5 L of blood carrying 750 g of hemoglobin (Hb). Half of the volume of blood is taken up by Hb. At 95% of saturation, hemoglobin transports 0.05 mole of O₂ per cycle. Each gram of Hb transports 1.34 mL of oxygen. An additional 1.5% of oxygen is dissolved in the serum. It takes one minute for the blood to circulate through the body.

A dose of 3.0 mg/L or higher of cyanide (CN) in the blood is considered to be fatal. Under these circumstances the oxygen saturation that normally is in excess of 95% drops to below 80%, which is insufficient to maintain cellular functioning. Oxygen saturation of the blood under these circumstances needs to be raised by 5% to maintain body functions.

Raising the oxygen saturation by 5% requires the equivalent of 50 mL of oxygen. One bag of blood, 500 mL, has the Hb content to supply 91 mL of oxygen. One bag of one of an exemplary perfluorocarbon, Oxygent, is equivalent up to two bags of blood in oxygen content.

Under load, blood vessels dilate, for example in pregnancy, typically a change of 48% in blood volume takes place with dilation of the blood vessels. The blood pressure (BP) declines, systolic by 3% and the diastolic by 24%.

The oxygen carried by the perfluorocarbon is rapidly dispersed and partially dissolved in the plasma. Due to the small size of the perfluorocarbon particles very small arterioles can be accessed. Using perfluorocarbon allows the infusion of an “overload” of oxygen, mimicking hyperbaric oxygen therapy delivery, increasing the dissolved oxygen to levels adequate for life. This provides an alternate pathway for the transport of oxygen to the tissues. The raised oxygen saturation also increases the cytochrome oxidase activity.

In a typical application, when the exposure level exceeds 3 mg/L of cyanide in the blood, infusion of 300 mL of a perfluorocarbon oxygen carrier is appropriate. In one example, the infusion rate is 500 mL/min. The blood volume increase due to the perfluorocarbon addition will raise the blood pressure by an estimated 20 mmHg. With the use of a relaxant that dilates the blood vessels a volume overload is not an issue. The volume increase also causes a temporary hypervolumic hyponatremia and hypokalemia resulting in a small electrolyte imbalance but without significant impact on the electrophysiology. In six to twelve hours the body clears the perfluorocarbons from the system.

Preferably, a patient is identified and selected for treatment according to an embodiment of an inventive method. For example, a patient in need of treatment may be an individual that is suffering from toxic agent poisoning such that an increase in myocardial contractility with reduced energy requirements is an intended therapy and where inhibition of toxic agent-caused distortion of the action potential of myocytes and support of restoration of usual intracellular ionic concentrations is desirable.

As noted above, toxic agents, such as cyanide, are in common use in particular industrial settings, as well as being a hazard encountered on the battlefield or in terrorist situations. Thus, an individual in need of treatment for toxic agent poisoning may be characterized by an occupation likely to bring the individual in contact with a toxic agent and/or history of toxic agent exposure. Often of interest in identifying an individual in need of treatment is a history of the individual's recent activity or location, presence in a war zone or area known for terrorist activity being an indicator making exposure to a toxic agent more likely.

Toxic agent poisoning may occur by any of various routes illustratively including ingestion, inhalation, skin exposure, mucosal exposure, and parenteral exposure.

Cyanide exposure leading to toxic effects may be in any of various forms such as exposure to cyanide containing gases or liquids including hydrogen cyanide and/or cyanogen chloride. Toxic effects may further be linked to exposure to cyanide containing compounds illustratively including potassium cyanide, sodium cyanide, ammonium sulfocyanide, potassium ferricyanide, potassium ferrocyanide, sodium nitroprusside, and complex cyanides such as fulminates, cyanates and thiocyanates.

Symptoms and signs of cyanide poisoning include early symptoms such as anxiety, headache, vertigo, hyperpnea, dyspnea, hypertension, bradycardia and cardiac arrhythmias such as sinus or AV nodal arrhythmias. Further symptoms include loss of consciousness, convulsions and cardiac arrest.

Of particular interest in the context of an embodiment of an inventive method are cardiovascular symptoms including electrocardiography changes such as atrial fibrillation, entopic ventricular heartbeats, abnormal QRS complex and sinus bradycardia.

Elevated blood cyanide concentration and urinary presence of thiocyanate can confirm exposure to cyanide. Acidosis and elevated venous blood oxygen are also common laboratory findings. However, where likelihood of cyanide exposure is present, rapid treatment is required since death can occur in less than 10 minutes in severe cases.

Thus, in one embodiment, an individual selected for treatment with an inventive composition and/or method is suffering from toxic agent-caused heart failure and/or a toxic agent-caused contractility deficit. Such an individual may have a history or likelihood of toxic agent exposure and symptoms of toxic agent-caused heart failure and/or a toxic agent-caused contractility deficit. Optionally, toxic agent exposure is confirmed by assay for a toxic agent, a toxic agent metabolite, or a clinical and/or pathophysiological finding consistent with toxic agent exposure.

The methods of the invention include both acute and chronic therapies. Relatively long-term administration of a therapeutic agent also will be beneficial after a patient has suffered from chronic toxic agent-caused heart failure to provide increased exercise tolerance and functional capacity. A chloride conductance inhibitor, such as DIDS, can be administered to a patient after having suffered heart failure due to toxic agent-caused toxicity to promote enhanced functional capacity.

For example, DIDS can be immediately administered to a patient, e.g. intravenously or intraperitonially, that has suffered or is suffering from congestive heart failure or cardiogenic shock. Such immediate administration preferably would entail administration of a therapeutic agent within minutes after a subject exhibits a symptom and/or sign of toxic agent-caused ventricular fibrillation or cardiogenic shock. In one embodiment, an oral dosage is preferred.

A patient in need of treatment for toxic agent poisoning is discussed herein in general terms relating to human individuals. However, inventive methods and compositions for individuals of other species, particularly mammalian species such as non-human primates, sheep, dogs, cats, cattle, pigs, horses and the like, are considered to be within the scope of the invention.

While compositions and methods according to the invention are described as relating to treatment of toxic agent-caused cardiac symptoms and signs, it is appreciated that other cells and tissues may be affected by a toxic agent, particularly cyanide, and that compositions and methods described herein may be applicable to treatment of those other cells and tissues. In particular, excitable membranes of cells such as neurons may have membrane potentials and ion concentrations across those membranes are distorted as a result of toxic agent poisoning and it is appreciated that inventive methods and compositions may be used to treat such sequelae of toxic agent exposure.

In one embodiment, an inventive pharmaceutical composition includes a modulator of a biological cell membrane conductance as described herein and a pharmaceutically acceptable carrier. The term “pharmaceutically acceptable carrier” as used herein is intended to refer to a carrier or diluent that is generally non-toxic to an intended recipient and which does not significantly inhibit activity of an active agent included in the composition.

In one embodiment, a composition according to the present invention is provided which includes at least one of the following: a chloride current modulator and an exogenous oxygen carrier. In one embodiment, an inventive composition preferably further includes a therapeutic agent to inhibit poison-caused pathology. In a further preferred embodiment, the chloride current modulator is an inhibitor of ICl, swell.

An inventive composition is suitable for administration to patients by a variety of systemic and/or local routes illustratively including intravenous, oral, parenteral, intrathecal, intraventricular, intracardiac, pericardiac, and mucosal.

An inventive composition may be administered acutely or chronically. For example, in an emergency situation, a conductance modulator included in a composition as described herein may be administered as a unitary dose or in multiple doses over a relatively limited period of time, such as seconds-hours. In a further embodiment, administration may include multiple doses administered over a period of days-years, such as for chronic treatment of long-lasting sequelae of toxic agent poisoning.

A therapeutically effective amount of a current modulator and of a therapeutic agent described herein will vary independently depending on the particular compound and on the particular agent used, the severity of the toxic agent exposure including the route of toxic agent exposure and the identity of the toxic agent, the length of time since toxic agent exposure and the general physical characteristics of the individual to be treated. One of skill in the art could determine a therapeutically effective amount in view of these and other considerations typical in medical practice. In general it is contemplated that a therapeutically effective amount would be in the range of about 0.001 mg/kg-100 mg/kg body weight, more preferably in the range of about 0.01-10 mg/kg, and further preferably in the range of about 0.1-5 mg/kg. Further, dosage may be adjusted depending on whether treatment is to be acute or continuing.

Compositions suitable for delivery may be formulated in various forms illustratively including physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, and vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of the required particle size in the case of dispersions, and by the use of surfactants, such as sodium lauryl sulfate. Such formulations are administered by a suitable route including parenteral and oral administration. Administration may include systemic or local injection, and particularly intravenous injection.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and substances similar in nature. Prolonged delivery of an injectable pharmaceutical form can be brought about by the use of agents delaying absorption, for example, aluminum monostearate and gelatin.

Solid dosage forms for oral administration include capsules, tablets, pills, powders, and granules. In such solid dosage forms, a conductance modulator is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, plant starches such as potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, glycerol monostearate, and glycols (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particularly preferred enteric coating material for use herein is those acrylic acid polymers and copolymers available under the trade name EUDRAGIT®, Roehm Pharma (Germany). The EUDRAGIT® series L, L-30D S copolymers, and cross-linked polymers, see for example U.S. Pat. No. 6,136,345, are most preferred since these are insoluble in stomach and dissolve in the intestine.

The enteric coating provides for controlled release of the active agent, such that release is accomplished at a predictable location in the lower intestinal tract below the point at which drug release would occur absent the enteric coating. The enteric coating also prevents exposure of the active agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated solid dosages of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The enteric coating optionally contains a plasticizer to prevent the formation of pores and cracks that allow the penetration of the gastric fluids into the solid dosage. Suitable plasticizers illustratively include, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The enteric coating is applied to a solid dosage using conventional coating methods and equipment. For example, an enteric coating can be applied to a solid dosage using a coating pan, an airless spray technique, fluidized bed coating equipment, or the like. Detailed information concerning materials, equipment and processes for preparing coated dosage forms may be found in Pharmaceutical Dosage Forms: Tablets, eds. H. A. Lieberman et al. (New York: Marcel Dekker, Inc., 1989), and in L. V. Allen, Jr. et al., Ansel's Pharmaceutical Dosage Forms and Drug Delivery Systems, 8th Ed. (Philadelphia, Pa.: Lippincott, Williams & Wilkins, 2004).

Liquid dosage forms for oral administration include a pharmaceutically acceptable carrier formulated as an emulsion, solution, suspension, syrup, or elixir. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to an inventive conjugate, may contain suspending agents, as for example, ethoxylated isostearyl alcohols, polyoxyethylene sorbitol and sorbitol esters, microcrystalline cellulose, aluminum metahydroxide, bentonite, agar-agar or tragacanth, or mixtures of these substances, and the like.

An ion channel modulator and/or therapeutic agent administered in an inventive method is defined herein as an ion channel modulator and/or therapeutic agent and pharmaceutically acceptable salts, derivatives, oxides and hydrates thereof. The term “pharmaceutically acceptable salt, derivative, oxide and hydrate” refers to a formulation that is substantially non-toxic to the individual being treated and which does not substantially inhibit the activity of an active agent being administered. The term “derivative” as used herein refers to a channel modulator and/or a therapeutic agent which is chemically modified to include a nitrogen, oxygen, carbon, sulfur, halogen or phosphorus containing moiety illustratively including a C₁-C₄ substituted or unsubstituted, straight chain or branched alkyl, an amine, a sulfhydryl and an oxide. The term “oxide” as used herein refers to an oxygen-containing derivative of a channel modulator and/or a therapeutic agent. Exemplary oxygen containing derivatives include an oxygen containing moiety such as a carboxyl, a carbonyl, a sulfonyl, a sulfoxy, a hydroxyl, a nitro, a phosphate, and a C₁-C₄ substituted or unsubstituted, straight chain or branched alkyl linked to the channel modulator and/or a therapeutic agent by ester or ether linkage.

Further examples and details of pharmacological formulations and ingredients are found in standard references such as: A. R. Gennaro, Remington: The Science and Practice of Pharmacy, Lippincott Williams & Wilkins, 20th ed. (2003); L. V. Allen, Jr. et al., Ansel's Pharmaceutical Dosage Forms and Drug Delivery Systems, 8th Ed. (Philadelphia, Pa.: Lippincott, Williams & Wilkins, 2004); J. G. Hardman et al., Goodman & Gilman's The Pharmacological Basis of Therapeutics, McGraw-Hill Professional, 10th ed. (2001).

While compositions and methods described herein are primarily described in the context of cyanide poisoning, it is appreciated that compositions and methods as described may be applicable to treatment of other types of poison exposure in which an inactive ion current is activated following exposure such that cell membrane potentials and ion concentrations across those membranes are distorted, symptoms and signs indicative of pathological excitable cell function. Further, an individual may be exposed to combinations of poisons for which inventive methods and compositions are applicable.

Embodiments of inventive compositions and methods are illustrated in the following examples. These examples are provided for illustrative purposes and are not considered limitations on the scope of inventive compositions and methods.

### EXAMPLE 1

An individual having symptoms of cyanide-caused cardiac arrhythmia is injected intravenously with 10 mg/kg 4,4′-diisothiocyanostilbene-2,2′-disulfonic acid (DIDS) dissolved in normal saline. The effect of the treatment may be monitored by ECG.

### EXAMPLE 2

An individual presents with symptoms of cyanide-caused cardiac arrhythmia. A solution of 20 mg/kg of indanyloxyacetic acid (IAA-94) is prepared by dissolving the IAA-94 in ethanol and then diluting the material to the final concentration in normal saline. The cyanide affected individual is injected intravenously with the IAA-94 preparation. The effect of the treatment may be monitored by ECG.

### EXAMPLE 3

An individual having symptoms of cyanide-caused cardiac arrhythmia is injected intravenously with 0.5 mg/kg of tamoxifen dissolved in ethanol and then diluted to final concentration in normal saline. The effect of the treatment may be monitored by ECG.

### EXAMPLE 4

An individual having symptoms of cyanide-caused cardiac arrhythmia is injected intravenously with 1 mg/kg of 5-nitro-2-(3-phenylpropylamino) benzoic acid (NPPB) dissolved in ethanol and then diluted to final concentration in normal saline. The effect of the treatment may be monitored by ECG.

### EXAMPLE 5

An individual having a history of cyanide exposure and symptoms of cardiac rhythm abnormalities ingests 25 mg/kg of 4,4′-diisothiocyanostilbene-2,2′-disulfonic acid (DIDS) in tablet form. The effect of the treatment may be monitored by ECG.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating a poison-caused pathology in an individual subject exposed to a poison, comprising:
administering a pharmaceutical composition comprising a therapeutically effective amount of at least one of: a chloride current modulator and an exogenous oxygen carrier, to an individual subject exposed to a poison, the therapeutically effective amount effective to reduce a symptom or sign of a poison-caused pathology, thereby treating the poison-caused pathology.

2. The method of claim 1 wherein the chloride current modulator is a modulator of ICl, swell.

3. The method of claim 1 wherein the chloride current modulator is selected from the group consisting of: a disulfonic stilbene, an arylaminobenzoate, a fenamate, an anthracene carboxylate, an indanylalkanoic acid, clofibric acid, a clofibric acid derivative, a sulfonylurea, a calixarene, suramin, and tamoxifen.

4. The method of claim 2 wherein the modulator of ICl, swell is selected from the group consisting of 4,4′-diisothiocyanostilbene-2,2′-disulfonic acid (DIDS); 4,4′,-dinitrostilbene-2,2 ′-disulfonic acid (DNDS); 4-acetamido-4′-isothiocyanostilbene-2,2′-disulfonic acid (SITS); tamoxifen, 5-nitro-2-(3-phenylpropylamino) benzoic acid (NPPB); nifiumic acid (NFA); flufenarnic acid; anthracene-9-carboxylate (9AC); diphenylaminecarboxylate (DPC), 2-(p-chlorophenoxy)propionic acid (CPP); and indanyloxyacetic acid (IAA-94).

5. The method of claim 1 wherein the exogenous oxygen earner is selected from the group consisting of a hemoglobin-based exogenous oxygen carrier and a fluorocarbon-based exogenous oxygen earner.

6. The method of claim 1 further comprising administering a therapeutic agent to inhibit poison-caused pathology.

7. The method of claim 6 wherein the therapeutic agent is a second current modulator.

8. The method of claim 6 wherein the therapeutic agent is an antiarrhythmic drug.

9. The method of claim 6 wherein the therapeutic agent is a modulator of a mitochondrial membrane moiety selected from the group consisting of: an ion channel, an ion pump, an ion exchanger, and a combination thereof.

10. The method of claim 6 wherein the therapeutic agent is an inhibitor of protein kinase C.

11. The method of claim 1 wherein the poison is selected from the group consisting of: a toxic agent, a chemical asphyxiant, and a combination thereof.

12. The method of claim 11 wherein the toxic agent is selected from the group consisting of a cyanide, an azide, carbon monoxide, and a combination thereof.

13. The method of claim 11 wherein the chemical asphyxiant is selected from the group consisting of a toxic agent, hydrogen sulfide, nitrogen oxide, chlorine and a combination thereof.

14. The method of claim 6 wherein the therapeutic agent is a cyanide clearing agent.

15. The method of claim 14 wherein the cyanide clearing agent is selected from the group consisting of: a sulfur donor, a methemoglobin former, a cyanide binding agent; and a combination thereof.

16. The method of claim 1 wherein the poison-caused pathology is a cardiac abnormality.

17. The method of claim 16 wherein the cardiac abnormality is selected from the group consisting of: ventricular fibrillation and cardiogenic shock.

18. The method of claim 7 wherein the second current modulator is a sodium channel opener.

19. The method of claim 18 wherein the sodium channel opener is selected from the group consisting of: veratridine, aconitine, and a combination thereof.

20. The method of claim 7 wherein the second current modulator is a calcium channel opener.

21. A quick acting therapeutic composition to mitigate and modulate cyanide poisoning consisting of:
the combination of: a chloride current modulator to rapidly block a cyanide anion; an exogenous oxygen carrier; and a therapeutic agent to inhibit cyanide poison-caused pathology.

22. The composition of claim 21 wherein the chloride current modulator is a modulator of ICl, swell.

23. The composition of claim 21 wherein the exogenous oxygen carrier is selected from the group consisting of a hemoglobin-based oxygen carrier and a fluorocarbon-based carrier.

24. The composition of claim 21 wherein the therapeutic agent is selected from the group consisting of: a second current modulator, an antiarrhythmic drug, a toxic agent-clearing agent, an inhibitor of protein kinase C, a modulator of a mitochondrial membrane moiety, and a combination thereof.

25. A composition of claim 21 wherein the poison-caused pathology is a cardiac abnormality.

26. The composition of claim 21 wherein the exogenous oxygen carrier comprises a fluorocarbon oxygen carrier dispersed in an aqueous phase and wherein the therapeutic agent is present in the aqueous phase.
